(19) United States
(12) Patent Application Publication
(10) Pub. No.: US 2024/0245927 A1
(43) Pub. Date: Jul. 25, 2024

(54) ULTRAVIOLET THERAPY APPARATUS AND LIGHT SOURCE

(71) Applicant: USHIO DENKI KABUSHIKI KAISHA, Tokyo (JP)

(72) Inventor: Hiroshi SHIBATA, Tokyo (JP)

(21) Appl. No.: 18/577,616

(22) PCT Filed: Mar. 25, 2022
(86) PCT No.: PCT/JP2022/014373

§ 371 Date: Jan. 8, 2024

(30) Foreign Application Priority Data
Jul. 19, 2021 (JP) 2021-118836

(51) Int. Cl. A61N 5/06

(52) U.S. Cl. CPC A61N 5/0616; A61N 2005/0644; A61N 2005/0651; A61N 2005/0661

(57) ABSTRACT

An ultraviolet therapy apparatus is provided and includes a light source unit that emits ultraviolet light, the light source unit comprising at least one LED, the LED being configured to, in a case in which an integral intensity of an emission spectrum in a wavelength range of 250 nm to 400 nm is 1, have a ratio of an integral intensity in wavelengths of 250 nm to 298 nm to an integral intensity in the wavelength range is less than or equal to 0.088, and emit an emission spectrum such that a ratio of an integral intensity of wavelengths of 308 nm to 313 nm to an integral intensity of wavelengths of 250 nm to 298 nm is greater than or equal to 5.2.

### CROSS REFERENCE TO RELATED APPLICATIONS

This application is a U.S. National Phase application under 35 U.S.C. 371 of International Application No. PCT/JP2022/014373, filed on Mar. 25, 2022, which claims priority to Japanese Patent Application No. 2021-118836, filed on Jul. 19, 2021. The entire disclosures of the above applications are expressly incorporated by reference herein.

### BACKGROUND

### Technical Field

This invention relates to ultraviolet therapy technology that use LEDs for light sources.

### Related Art

Conventional phototherapy includes ultraviolet therapy that uses ultraviolet light in the wavelength range of UVA (wavelength of 320 nm to 400 nm) and UVB (wavelength of 280 nm to 320 nm). Ultraviolet therapy is a treatment that involves immunosuppression through ultraviolet irradiation to achieve therapeutic effects.

For example, JP-2010-5438A discloses an ultraviolet therapy apparatus that treats skin diseases with ultraviolet light, where the ultraviolet therapy apparatus includes a light source lamp as an ultraviolet light source.

On the other hand, the development of LEDs (Light Emitting Diodes) has been remarkable in recent years, and not only in general lighting, but also in many industrial machines and equipment, switching of light sources from lamps to LEDs is progressing. In addition, LEDs are becoming more powerful not only in the visible light range but also in the ultraviolet range, and LEDs are expected to be a light source in the medical field as well.

For example, fluorescent lamps, metal halide lamps, and excimer lamps are used as light sources in phototherapy apparatus that uses mid-wave ultraviolet light in the wavelength range of 308 nm to 313 nm, but recently, an ultraviolet therapy apparatus that uses ultraviolet light emitting devices (UVLED) using LEDs as a light source.

When LEDs are used as a light source, the circuit configuration can generally be simpler than that for a power supply of a lamp, and the apparatus can be made smaller and lighter.

In the following description, ultraviolet light and light containing ultraviolet light are sometimes simply referred to as “light”.

The effects of ultraviolet light in the wavelength ranges such as UVA and UVB on a skin vary depending on the wavelength of the ultraviolet light. Generally, in the ultraviolet therapy apparatus, light is irradiated to the affected area within a range that does not cause side effects, depending on the wavelength of light emitted from the light source, in order to achieve therapeutic effects.

A conventional ultraviolet therapy apparatus which uses excimer lamps as a light source that emit light with a peak wavelength of 308 nm in the emission spectrum (radiation spectrum) is known. However, when LEDs with a peak wavelength equivalent to that of the excimer lamps are used as the light source, therapeutic effects may be lower or side effects (occurrence of erythema) may increase compared to a conventional ultraviolet therapy apparatus that uses the excimer lamps.

Considering the above issue, it is an object of the present invention to provide a light source that includes LEDs which can achieve at least one of the same or better therapeutic effects and the same or worse side effects compared to excimer lamps, and an ultraviolet therapy apparatus that uses the light source.

### SUMMARY

In order to solve the above problem, one aspect of an ultraviolet therapy apparatus according to the present invention is an ultraviolet therapy apparatus, including a light source unit that emits ultraviolet light, the light source unit comprising at least one LED, the LED being configured to: in a case in which an integral intensity of an emission spectrum in a wavelength range of 250 nm to 400 nm is 1, have a ratio of an integral intensity in wavelengths of 250 nm to 298 nm to an integral intensity in the wavelength range is less than or equal to 0.088; and emit an emission spectrum such that a ratio of an integral intensity of wavelengths of 308 nm to 313 nm to an integral intensity of wavelengths 250 nm to 298 nm is greater than or equal to 5.2.

In this case, it is possible to provide the ultraviolet therapy apparatus that uses LEDs as the light source that achieves the same or better therapeutic effect and the same or lower risk of side effects compared to a conventional ultraviolet therapy apparatus that uses excimer lamps as the light source.

Further, one aspect of an ultraviolet therapy apparatus according to the present invention is an ultraviolet therapy apparatus, including a light source unit that emits ultraviolet light, the light source unit comprising at least one LED, the LED being configured to: in a case in which an integral intensity of an emission spectrum in a wavelength range of 250 nm to 400 nm is 1, have an intensity at a wavelength of 298 nm is less than or equal to 0.0078; have an intensity at a wavelength of 295 nm is less than or equal to 0.0055; have an intensity at a wavelength of 290 nm is less than or equal to 0.0033; have an intensity at a wavelength of 280 nm is less than or equal to 0.0015; and emit an emission spectrum such that a ratio of an integral intensity of wavelengths of 308 nm to 313 nm to an integral intensity of wavelengths 250 nm to 298 nm is greater than or equal to 5.2.

In this case, it is possible to provide the ultraviolet therapy apparatus that uses LEDs as the light source that achieves the same or better therapeutic effect and the same or lower risk of side effects compared to a conventional ultraviolet therapy apparatus that uses excimer lamps as the light source.

Further, one aspect of an ultraviolet therapy apparatus according to the present invention is an ultraviolet therapy apparatus, including a light source unit that emits ultraviolet light, the light source unit comprising at least one LED, the LED being configured to: in an erythema ultraviolet spectrum E_{CIE} in a wavelength range of 250 nm to 400 nm, emit an emission spectrum such that a ratio of an integral intensity of wavelengths of 250 nm to 298 nm to an integral intensity of the wavelength range is less than or equal to 0.44, wherein the erythema ultraviolet spectrum E_{CIE} is represented as

$${E}_{CIE} = {E}_{λ} \times {S}_{er} / ( {\int }_{250}^{400} {E}_{λ} d λ )$$

where E_{λ} is a spectral irradiance of the ultraviolet light emitted from the LED, and Sₑᵣ is an erythema action spectrum and represented as

$${S}_{e r} ( λ ) = \{ \begin{matrix}1 & ( 250 nm < λ < 298 nm ) \\ 1 {0}^{0.094 ( 298 - λ )} & ( 298 nm ≦ λ ≦ 328 nm ) \\ 1 {0}^{0.015 ( 139 - λ )} & ( 328 nm < λ < 400 nm ) .\end{matrix}$$

Further, the erythema ultraviolet spectrum E_{CIE} is represented as

$${E}_{CIE} = P \times {S}_{e r}$$

where P is an emission spectrum of the LED, area normalized in a wavelength range of 250 nm to 400 nm.

Further, the above-described ultraviolet therapy apparatus may be configured to emit an emission spectrum such that a ratio of an integral value of wavelengths of 308 nm to 313 nm to an integral value of wavelengths of 250 nm and 298 nm is greater than or equal 0.47 in the erythema ultraviolet spectrum E_{CIE}.

In this case, it is possible to provide the ultraviolet therapy apparatus that uses LEDs as the light source that achieves the same or better therapeutic effect and the same or lower risk of side effects compared to a conventional ultraviolet therapy apparatus that uses excimer lamps as the light source.

Further, in the above-described ultraviolet therapy apparatus, a peak wavelength of an emission spectrum of the LED may be between 308 nm and 313 nm. In this case, the ultraviolet therapy apparatus is more suitable for mid-wave ultraviolet therapy.

Further, in the above-described ultraviolet therapy apparatus, a full width at half maximum of an emission spectrum of the LED may be less than or equal to 20 nm. In this case, it is possible to provide the ultraviolet therapy apparatus that uses LEDs with an emission spectrum with low risk of side effects as the light source.

Further, the above-described ultraviolet therapy apparatus may be configured to emit (receive) light having the spectrum of the above-described feature on an irradiated surface.

In this case, it is possible to emit the light having the same or similar spectral characteristics as the light source on the irradiated surface, and an improvement in therapeutic effect and/or a reduction in side effects may be confirmed on the irradiated surface compared to the conventional apparatus.

Further, in order to solve the above problem, one aspect of a light source according to the present invention is a light source including LED that emits the above-described emission spectrum.

In this case, it is possible to provide a light source equipped with LEDs and an ultraviolet therapy apparatus that uses the light source, which can achieve at least one of the same or better therapeutic effect and the same or worse side effects compared to excimer lamps.

### Advantageous Effects of the Invention

According to the present invention, it is possible to provide a light source that includes LEDs which can achieve at least one of the same or better therapeutic effects and the same or worse side effects compared to excimer lamps, and an ultraviolet therapy apparatus that uses the light source.

### BRIEF DESCRIPTION OF THE DRAWINGS

FIG. 1 is a graph showing the erythema action spectrum defined by the CIE.

FIG. 2A is a graph showing characteristics of the emission spectrum of excimer lamps.

FIG. 2B is an enlarged graph of FIG. 2A, showing characteristics of the emission spectrum of excimer lamps.

FIG. 3A is a graph showing characteristics of the erythema action spectrum defined by the CIE and the emission spectrum of excimer lamps.

FIG. 3B is a graph of the erythema ultraviolet spectrum integral value E_{CIE}.

FIG. 4 is a block diagram of an example configuration of the ultraviolet therapy apparatus.

FIG. 5 is an oblique view of an example configuration of the treatment tool.

FIG. 6 is a front view of an example configuration of the treatment tool.

FIG. 7 is a cross-sectional view of an example internal configuration of the treatment tool.

### DETAILED DESCRIPTION

Hereinafter, with reference to the accompanying drawings, an embodiment of the present invention will be described.

In the present embodiment, an ultraviolet therapy apparatus which comprises a treatment tool having a light source that emits light containing ultraviolet light, for example, ultraviolet light in the range of UVB (wavelength of 280 nm to 320 nm) will be described.

### Relation Between Wavelength of Light and Susceptibility to Erythema

Exposure of human skin to ultraviolet light in the range of UVB may cause erythema as a side effect. Erythema is redness of the skin surface caused by dilation of capillaries. The minimal ultraviolet radiation dose that causes erythema on the skin is referred to as the minimal erythema dose (MED). The unit of MED is mJ/cm². In the same way as the susceptibility to sunburn varies among individuals, the susceptibility to erythema, or MED varies among individuals.

The susceptibility to ultraviolet erythema, i.e., the influence of ultraviolet on human bodies varies depending on the wavelength of the ultraviolet. The relative effectiveness on human bodies depending on the wavelength is defined by the International Commission on Illumination (CIE) as the erythema reference action spectrum.

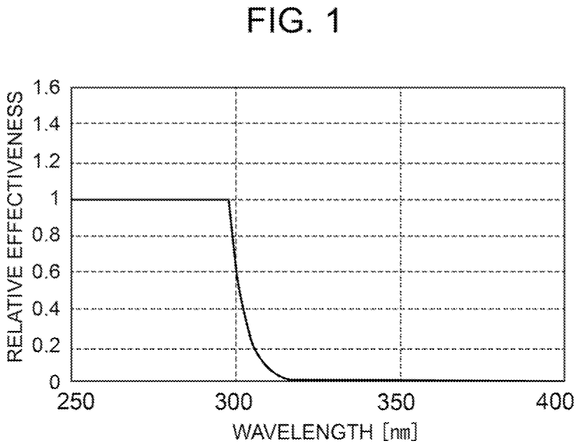

FIG. 1 is a graph showing the erythema action spectrum Sₑᵣ.

In FIG. 1, the horizontal axis indicates the wavelength λ [nm], and the vertical axis indicates the relative effectiveness. The erythema action spectrum Sₑᵣ is defined in the range of 250 nm to 400 nm, and is defined as a relative effectiveness depending on wavelengths as in Formula (1), in which the relative effectiveness is a relative value on the assumption that the influence of light with a wavelength of 250 nm to 298 nm on the human skin is 1.

$${S}_{e r} ( λ ) = \{ \begin{matrix}1 & ( 250 nm < λ < 298 nm ) \\ 1 {0}^{0.094 ( 298 - λ )} & ( 298 nm ≦ λ ≦ 328 nm ) \\ 1 {0}^{0.015 ( 139 - λ )} & ( 328 nm < λ < 400 nm )\end{matrix} \qquad ( 1 )$$

From the outline of the graph shown in FIG. 1, it can be understood that shorter wavelengths have a greater impact on human bodies and are more likely to cause erythema (higher risk of side effects). Specifically, light with wavelengths longer than the UVB region, or longer than the wavelength of 328 nm in a case in which Formula (1) is strictly applied, has little effect on the skin. On the other hand, light with wavelengths below 328 nm begins to affect the skin, and the effect increases with shorter wavelengths. Light with wavelength below 298 nm has the greatest effect (i.e., susceptibility to erythema) on human bodies. In other words, light with wavelengths below 298 nm has the highest risk of side effects on human bodies.

On the other hand, the inventors made a prototype ultraviolet therapy apparatus using LEDs with a peak wavelength equivalent to that of excimer lamps as a light source, and they found that even LEDs with a peak wavelength of approximately 308 nm had different symmetries on the long wavelength side and the short wavelength side centered on the peak wavelength and different half-widths of the peak wavelength depending on the company that manufactured the LEDs, or even on lot differences (individual differences) of the LEDs even if the same company produced them. For this reason, they found that even when LEDs with a peak wavelength of about 308 nm are used, therapeutic effect is lower and there are more side effects (erythema occurrences) than the conventional ultraviolet therapy apparatus that uses the excimer lamps.

From the above points, considering the characteristics of the erythema action spectrum shown in FIG. 1, the present embodiment intends to select LEDs so that they have the same or better therapeutic effects and/or the same or lower side effects compared to the excimer lamps and configure the ultraviolet therapy apparatus using the selected LEDs. In the following, an embodiment for selecting LEDs to be used in the ultraviolet therapy apparatus considering the characteristics of the excimer lamps will be described.

### LED Selection Criteria Considering Characteristics of Excimer Lamps

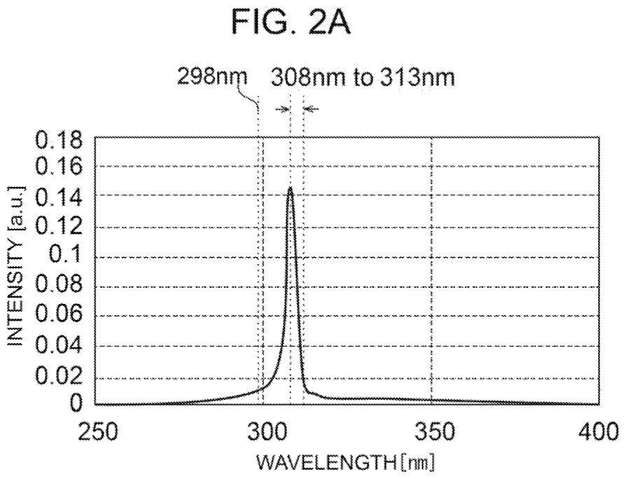

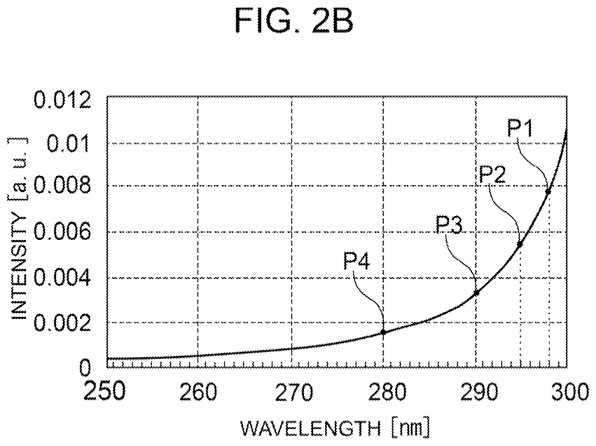

FIGS. 2A and 2B show graphs showing characteristics of the emission spectrum of excimer lamps with a peak wavelength of 308 nm. FIG. 2A shows a graph of the emission spectrum of excimer lamps with a wavelength of 250 nm to 400 nm. The vertical axis indicates the emission spectrum (intensity) of a wavelength of 250 nm and 400 nm, normalized using the total emission spectrum of the entire wavelength range (250 nm to 400 nm). The normalized emission spectrum is also referred to as an area-normalized emission spectrum. In the area-normalized emission spectrum, the integral intensity of the entire wavelength range (the area occupied by the area-normalized emission spectrum for a given wavelength range) is 1.

FIG. 2B shows an enlarged graph of the area below 300 nm wavelength in the graph in FIG. 2A.

From the graphs in FIGS. 2A and 2B, it can be understood that excimer lamps with a peak wavelength of 308 nm emit light with a wavelength of 298 nm or less, which has the greatest impact on human bodies.

Also, the inventors confirmed from the graphs in FIGS. 2A and 2B that the integral intensity in a wavelength of 250 nm to 298 nm accounts for 8.8% of the integral intensity in the entire wavelength range (250 nm to 400 nm), that is, the ratio of the integral intensity in a wavelength of 250 nm to 298 nm over the entire wavelength range is 0.088.

Therefore, it is confirmed that it is sufficient select LED so that the ratio of the integral intensity in a wavelength of 250 nm to 298 nm to the integral intensity in the entire wavelength range that LED can emit (for example, a wavelength range including a wavelength of 250 nm to 400 nm) is less than 0.088 (Criterion 1). Accordingly, it is possible to select LED that can emit less light of the short wavelength component below 298 nm, which has the highest risk of side effects, than excimer lamps.

Furthermore, while Criterion 1 uses the wavelength range of 250 nm to 298 nm as a selection index, selection criteria in terms of a single wavelength index for LED that can emit less light of the short wavelength component below 298 nm, which has highest risk of side effects, than excimer lamps, is assumed.

From the graphs in FIGS. 2A and 2B, it can be understood that, for excimer lamps, the area-normalized spectral intensity at a wavelength of 298 nm is 0.0078 (corresponding to the point P1 in FIG. 2B), the area-normalized spectral intensity at a wavelength of 295 nm is 0.0055 (corresponding to the point P2 in FIG. 2B), the area-normalized spectral intensity at a wavelength of 290 nm is 0.0033 (corresponding to the point P3 in FIG. 2B), and the area-normalized spectral intensity at a wavelength of 280 nm is 0.0015 (corresponding to the point P4 in FIG. 2B).

Therefore, as the selection criterion for LED that can emit less light of the shorter wavelength component below 298 nm, which has the highest risk of side effects, than excimer lamps, Criterion 2 that meets the following (1), (2), (3), and (4) can be set instead of, or in addition to Criterion 1.
(1) In the area-normalized emission spectrum of LED, the intensity at a wavelength of 298 nm is less than or equal to 0.0078 (corresponding to the point P1 in FIG. 2B).
(2) In the area-normalized emission spectrum of LED, the intensity at a wavelength of 295 nm is less than or equal to 0.0055 (corresponding to the point P2 in FIG. 2B).
(3) In the area-normalized emission spectrum of LED, the intensity at a wavelength of 290 nm is less than or equal to 0.0033 (corresponding to the point P3 in FIG. 2B).
(4) In the area-normalized emission spectrum of LED, the intensity at a wavelength of 280 nm is less than or equal to 0.0015 (corresponding to the point P4 in FIG. 2B).

In other words, it is sufficient that the emission spectrum has an intensity characteristic less than or equal to the graph (line (curve) connecting each point) passing through points P1 to P4 in FIG. 2B in a wavelength of 250 nm to 300 nm.

In mid-wave ultraviolet therapy, light in the wavelength range of 308 nm to 313 nm is used for treatment. The inventors confirmed from the graph shown in FIG. 2A that the integral intensity in the wavelength range of 308 nm to 313 nm accounts for 46% of the integral intensity in the entire wavelength range (250 nm to 400 nm) of the light emitted from the lamp.

As mentioned above, in the case of excimer lamps, the integral intensity in the wavelength of 250 nm to 298 nm accounts for 8.8% of the integral intensity in the entire wavelength range (250 nm to 400 nm).

Therefore, considering the balance between the risk of side effects and therapeutic effects, it is effective to select LED having the emission spectrum in which the integral intensity in the wavelength of 250 nm to 298 nm is less than or equal to 8.8%, and the integral intensity in the wavelength of 308 nm to 313 nm is greater than or equal to 46.0%, over the entire wavelength range (wavelength of 250 nm to 400 nm).

In other words, it is sufficient to select LED in which the ratio of the integral intensity of the emission spectrum in the wavelength range of 308 nm to 313 nm to the integral intensity in the wavelength range of 250 nm to 298 nm is greater than or equal to 5.2 (=46.0/8.8) (Criterion 3). By using such selected LEDs, it is possible to configure a therapy apparatus with less risk of side effects and higher therapeutic efficacy than when using excimer lamps.

Considering the above, selection of LED that meets Criteria 1 and 3, or Criteria 2 and 3 makes it possible to provide the ultraviolet therapy apparatus that has the same or lower risk of side effects and the same or higher therapeutic effects compared to using excimer lamps as the light source.

In view of reducing the risk of side effects, Criterion 2 may be an additional criterion to Criterion 1. In other words, LED that meets Criteria 1, 2, and 3 may be selected.

Next, effects of ultraviolet radiation on the human body will be quantitatively considered.

The overall effect of ultraviolet radiation on the human body is obtained by integrating the product of the spectral irradiance (irradiance per wavelength of light) E_{λ} of the irradiated ultraviolet light and the erythema action spectrum Sₑᵣ in wavelengths of 250 nm to 400 nm. The erythema action spectrum Sₑᵣ is shown in Formula (1) and FIG. 1.

The effect on the human body determined in this way is referred to as an erythemal ultraviolet dose I_{CIE} and is represented as in Formula (2) below.

$${I}_{CIE} = {\int }_{250}^{400} {E}_{λ} \times {S}_{e r} d λ \qquad ( 2 )$$

The higher the value of the erythemal ultraviolet dose I_{CIE}, the more likely the light is to cause erythema.

Next, the differences in susceptibility to erythema due to differences in the light source spectrum in the erythema ultraviolet dose I_{CIE} will be considered.

The erythema ultraviolet spectrum E_{CIE} which is obtained by multiplying the area-normalized spectral irradiance E_{λ} by the erythemal action spectrum Sₑᵣ, can be obtained as in Formula (3) as below in relation to Formula (2).

$${E}_{CIE} = {E}_{λ} \times {S}_{e r} / ( {\int }_{250}^{400} {E}_{λ} d λ ) = P \times {S}_{e r} \qquad ( 3 )$$

In Formula (3) above, P indicates the area-normalized light source spectrum.

Since the erythema action spectrum Sₑᵣ indicates the susceptibility to erythema due to the wavelength and the spectral irradiance E_{λ} indicates the intensity due to the wavelength, the erythema ultraviolet spectrum E_{CIE}, which is the value obtained by multiplying them, indicates the susceptibility to erythema (relative effect on the human body) for each wavelength of the light with the spectral irradiance E_{λ}. In other words, this means that the larger the integral value of the erythema ultraviolet spectrum E_{CIE}, the more likely the light is to cause erythema. Since the erythema ultraviolet spectrum E_{CIE} can be calculated based on the area-normalized light source spectrum, it can be obtained from any light source spectrum, not limited to the spectral irradiance E_{λ}.

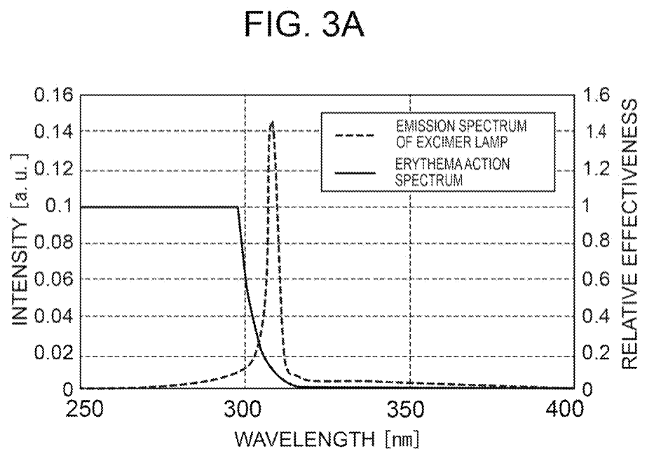

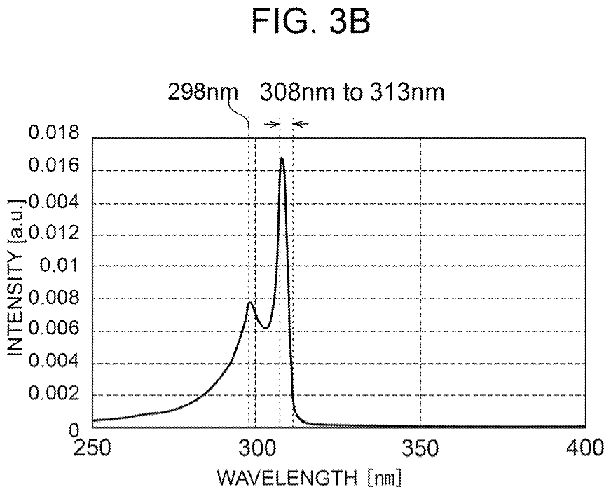

The characteristics indicated by Formula (3) will be described with reference to graphs. FIG. 3A shows a graph showing characteristics of the erythema action spectrum and the emission spectrum of excimer lamps, and FIG. 3B shows a graph of the erythema ultraviolet spectrum E_{CIE} shown in Formula (3) in excimer lamps.

In FIG. 3A, the solid line corresponds to the graph showing the erythema action spectrum shown in FIG. 1, and the dotted line corresponds to the area-normalized emission spectrum shown in FIG. 2A.

The graph of the area-normalized light source spectrum P in Formula (3) corresponds to the area-normalized emission spectrum shown in FIG. 2A. Therefore, the graph of the erythema ultraviolet spectrum E_{CIE} shown in FIG. 3B corresponds to the multiplication of the values shown by the solid and dotted graphs in FIG. 3A.

In order to reduce the risk of side effects compared to excimer lamps, referring to Formula (1) as above, it is required to select a light source with a lower erythemal ultraviolet dose in the wavelength range below 298 nm.

The inventors confirmed from FIG. 3B that, in the case of excimer lamps, the ratio of the integral value in the interval between 250 nm and 298 nm to the integral value in the interval between 250 nm and 400 nm in the erythema ultraviolet spectrum E_{CIE} is 0.44. Accordingly, it can be understood that it is sufficient to select LED in which the ratio is less than or equal to 0.44 in order to reduce the contribution of light, which has no therapeutic effects and the maximum risk of side effects, to a level equal to or greater than that of excimer lamps (Criterion 4).

In mid-wave ultraviolet therapy, the light in the wavelength range of 308 nm to 313 nm is used for treatment. As is clear from Formula (1) above, excimer lamps cause erythema effects even in the light in the wavelength range that is effective for treatment. By selecting a light source with a larger ratio of the erythematous effect of light with the wavelengths that have therapeutic effect (308 nm to 313 nm) to the erythematous effect of light with the wavelengths which are the main side effect (298 nm or less), it is possible to configure an apparatus with higher therapeutic effect (therapeutic efficiency).

To realize it in ratio to excimer lamps, it is sufficient to select LED such that, in the erythema ultraviolet spectrum E_{CIE}, the ratio of the integral value in 308 nm to 313 nm to the integral value in 250 nm to 298 nm is greater than or equal to 0.47 (Criterion 5).

In the above description, several criteria have been described for selecting LED to be used as the light source in the ultraviolet therapy apparatus. By using LED that meets Criteria 1, 2, 3, 4, or 5, it is possible to configure the ultraviolet therapy apparatus that achieves at least one of therapeutic effects equal to or better than that of excimer lamps and side effects equal to or lower than that of excimer lamps.

Hereafter, such LED selection criteria (the above single criteria or a combination of criteria) that can achieve at least one of therapeutic effects equal to or better than that of excimer lamps and side effects equal to or lower than that of excimer lamps is referred to as LED selection criteria.

In the above description, the emission spectrum of LED has been described. Alternatively, for example, a combination of an optical element such as a filter capable of filtering at least some wavelengths below 298 nm may be used as the light source. In other words, LEDs and such a filter may be used to configure an emission spectrum that meets the LED selection criteria.

In the above description, the selection of LED as the ultraviolet irradiation side has been mainly discussed. However, it is sufficient to configure an ultraviolet therapy apparatus that can emit (receive) light with a spectrum that meets the LED selection criteria on an irradiated surface, such as the skin of the human body. In other words, it is sufficient to configure an ultraviolet therapy apparatus so that the spectrum on the irradiated surface is a spectrum that meets the LED selection criteria. The spectrum at the light source unit of the ultraviolet therapy apparatus and the spectrum on the irradiated surface have the same or similar spectral characteristics due to the positional relationship between the ultraviolet therapy apparatus and the irradiated surface (see below) or the like. Therefore, by selecting LED that meets the LED selection criteria, the spectrum on the irradiated surface will also be the same (similar) as the emission spectrum.

In addition, the emission spectrum with peak wavelengths of 308 nm to 313 nm, among the emission spectrum that meets the LED selection criteria, may be selected. This enables the selection of LED more suitable for mid-wave ultraviolet therapy.

Furthermore, the emission spectrum with a full width at half maximum of 20 nm or less, among the emission spectra that meets the LED selection criteria, may be further selected. In other words, by using LED with emission spectrum broadening in a specified range of spectrum, it is possible to select LED that emit light with an emission spectrum with fewer short wavelength component below the wavelength of 298 nm that has the highest risk of side effects.

### Performance Evaluation

The performance evaluation of LED that meet the above LED selection criteria will be described. Generally, when LED is used as the light source in the ultraviolet therapy apparatus, a plurality of LEDs (e.g., about 20 to 30 LEDs) are used. In the present embodiment, the ultraviolet therapy apparatus is configured so that the light emitted from LEDs (or the light received on the irradiated surface) meets the above LED selection criteria. Therefore, not all of LEDs need to meet the LED selection criteria (it is sufficient that at least one LED meets the LED selection criteria so that the light emitted from the ultraviolet therapy apparatus meets the LED selection criteria). Alternatively, it is sufficient that the light emitted from the ultraviolet therapy apparatus meets the LED selection criteria on the irradiated surface.

TABLES 1A and 1B show the results of measuring the minimum irradiation dose (MED) at which erythema occurs in each of the ultraviolet therapy apparatus using LEDs that meet the LED selection criteria and the ultraviolet therapy apparatus using excimer lamps. Specifically, TABLES 1A and 1B show the evaluation results of the side effect resistance for the irradiation doses [mJ/cm²] in the case of using excimer lamps and LEDs as the light source of the ultraviolet therapy apparatus. The evaluation of side effect resistance is defined as “circle” which indicates good if no erythema occurs and “cross” which indicates bad if erythema occurs, when irradiated with a certain irradiation dose.

TABLE 1A
EXCIMER LAMP

| | | | |
|---|---|---|---|
| IRRADIATION DOSE [mJ/cm²] | 190 | 220 | 250 |
| SIDE EFFECT RESISTANCE | ◯ | X | X |

TABLE 1B
LED

| | | | |
|---|---|---|---|
| IRRADIATION DOSE [mJ/cm²] | 300 | 350 | 400 |
| SIDE EFFECT RESISTANCE | ◯ | ◯ | X |

From TABLE 1A, it can be understood that erythema is confirmed at 220 [mJ/cm²] when excimer lamps are used, and the MED is 220 mJ/cm². On the other hand, from TABLE 1B, it can be understood that erythema is confirmed at 400 [mJ/cm²] when LEDs that meet the LED selection criteria are used, and the MED is 400 mJ/cm².

From these results, it is confirmed that the use of LEDs selected based on the LED selection criteria provided less erythematous effects (side effects) than the use of conventional excimer lamps.

Next, the irradiation dose in the wavelength range effective for treatment (wavelength of 308 nm to 313 nm) of the MED determined from the results in TABLES 1A and 1B will be calculated.

The irradiation dose in the wavelength range effective for treatment is obtained from the MED determined by the evaluation results shown in TABLES 1A and 1B and the content ratio of the wavelength effective for treatment (wavelength of 308 nm to 313 nm) in the total wavelength range emitted. In the case of excimer lamps, the content ratio of the wavelength effective for treatment is 46%, as described above. In the case of LEDs used for the performance evaluation, it is assumed that the content ratio of the wavelength effective for treatment is 28%.

Under these conditions, the wavelength range effective for treatment in 1 MED in the case of excimer lamps can be calculated as

$$220 mJ / {cm}^{2} \times 0.46 = 101 mJ / {cm}^{2} .$$

Also, the wavelength range for treatment in 1 MED in the case of LEDs can be calculated as

$$400 mJ / {cm}^{2} \times 0.28 = 112 mJ / {cm}^{2} .$$

From the results, it could be confirmed that by selecting LEDs to meet the LED selection criteria, light in the wavelength range having therapeutic effects can be irradiated approximately 11% more than excimer lamps.

Therefore, by selecting LEDs as the light source based on the LED selection criteria described above, it is possible to realize the ultraviolet therapy apparatus capable of achieving at least one of a similar or lower risk of erythema (side effects) and a similar or higher therapeutic effects than before.

In addition, by using LEDs that meet the above criteria as the light source, it is possible to reduce the size and weight of the ultraviolet therapy apparatus while satisfying the same or better performance than conventional ones. Accordingly, it is expected to expand treatment applications and improve operability by operators. Furthermore, the use of LEDs is more energy-efficient than the use of lamps, which leads to a reduction in the amount of electricity used. Furthermore, the use of LEDs has the effect of extending the service life of the light source compared to the use of excimer lamps and reducing the frequency of light source replacement.

### Ultraviolet Therapy Apparatus Configuration

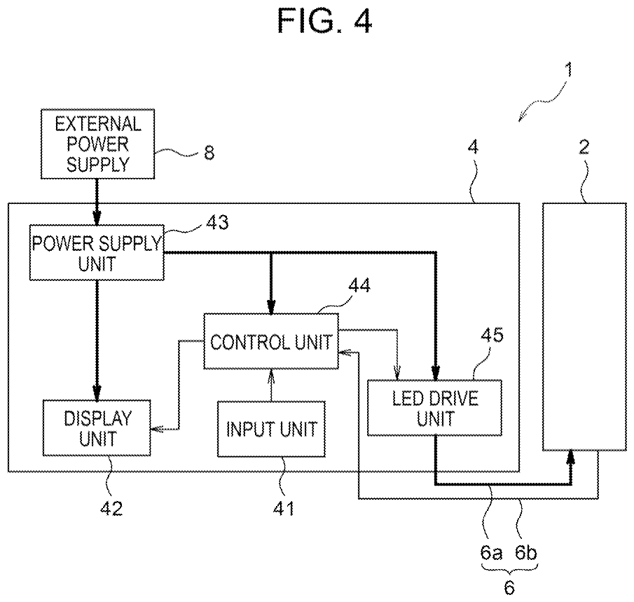

FIG. 4 is a block diagram showing an example configuration of the ultraviolet therapy apparatus 1.

The ultraviolet therapy apparatus 1 includes a treatment tool (light source unit) 2 including an LED light source that emits light containing ultraviolet light, and a main unit 4 that controls the LED light source in the treatment tool 2.

The main unit 4 includes an input unit 41, a display unit 42, a power supply unit 43, a control unit (controller) 44, and an LED drive unit 45. The treatment tool 2 and the main unit 4 are connected by a connection line 6 containing a power line 6a (shown in thick line) and a signal line 6b (shown in thin line).

The input unit 41 obtains the set irradiation dose entered by an operator (e.g., physician) and outputs its information to the control unit 44.

The display unit 42 can display information such as the intensity of the ultraviolet irradiation, the irradiation time, and the elapsed time during ultraviolet irradiation. The display 42 can also display information indicating that an abnormality has occurred (error messages, etc.) if some abnormality occurs in the ultraviolet therapy apparatus 1.

The power supply unit 43 converts power supplied from the external power supply 8 to an appropriate voltage for each unit for subsequent processing and supplies it to the unit.

The control unit 44 controls the LED drive unit 45 based on the information input from the input unit 41 to control the irradiation dose (irradiance or irradiation time) of the LED light source in the treatment tool 2.

The LED drive unit 45 supplies power to the LED light source in accordance with the control signal from the control unit 44.

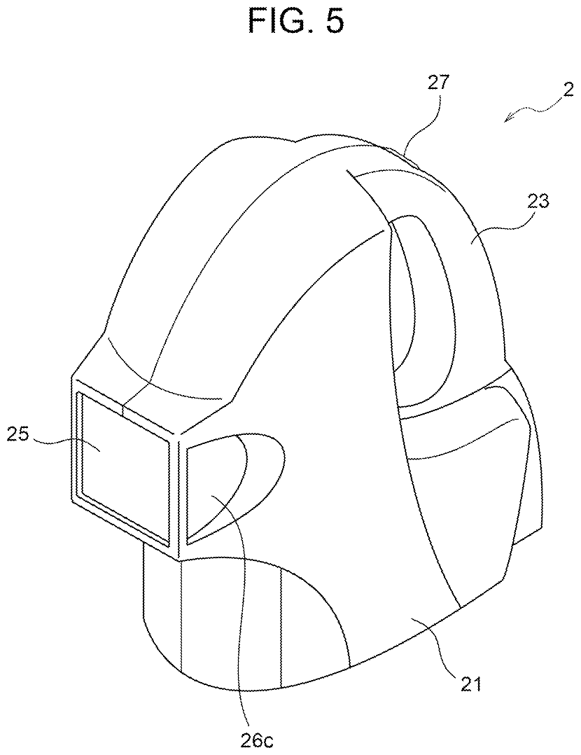

In the following, an example of the configuration of the treatment tool 2 will be described in detail with reference to FIGS. 5 to 7.

As shown in FIGS. 5 to 7, as an example, the treatment tool 2 includes a light source 24 that emits light containing ultraviolet light, a radiation window 25 for emitting light emitted from the light source 24 to the outside of the housing 21, a light guide unit 26 that guides light emitted from the light source 24 to the radiation window 25, and an instruction unit 27 for instructing the operator to turn on the light source 24.

The light source 24 is housed in a housing 21. The light source 24 includes a plurality of LEDs (UV LEDs) 24a that emit ultraviolet light. UV LEDs 24a are mounted on an LED substrate 24b.

At least one of the plurality of UV LEDs 24a is configured to meet the LED selection criteria so that the light emitted from the plurality of UV LEDs 24a (or the light received on the irradiated surface) meets the LED selection criteria. UV LEDs 24a emit ultraviolet light with wavelengths appropriate for the skin disease to be treated.

The light guide unit 26 includes a cylindrical reflector 26a in which the light source 24 is arranged inside, and a light guideway 26b that guides the light emitted from the light source 24 and the light reflected on the inner surface of the reflector 26a to the radiation window 25. A viewing window 26c is formed in a portion of the light guide 26b to allow viewing of the interior of the light guide 26b.

The operator can view the affected area from the viewing window 26c through the radiation window 25.

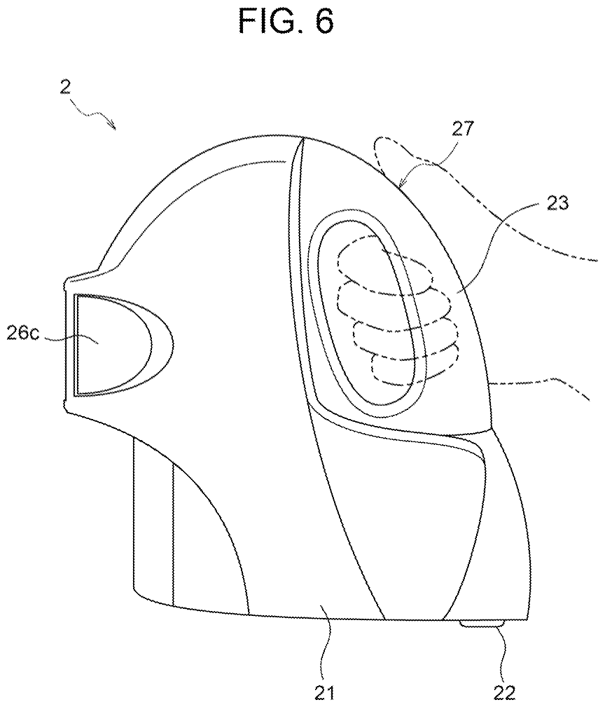

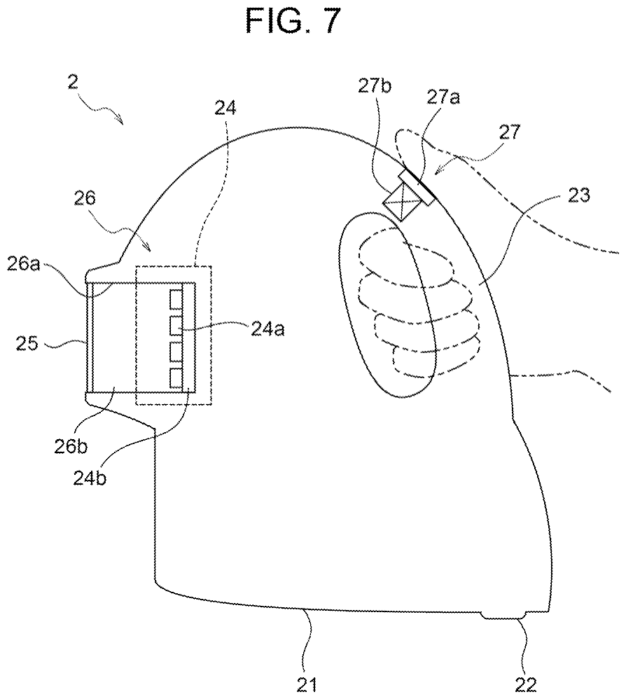

The indication unit 27 is arranged on one side of the gripping unit 23 (upper side in FIGS. 6 and 7). Specifically, the indication unit 27 is arranged on one side of the gripping unit 23 so that the operator can operate it with the thumb of the hand holding the gripping unit 23. For example, the indication unit 27 may be a push button switch which includes a button 27a to be contacted by the operator, and a force agent (e.g., a spring) 27b to be force-applied to the button 27a.

In the following, a procedure for the operator to irradiate the affected area with ultraviolet light using the ultraviolet therapy apparatus 1 according to the present embodiment will be described.

First, the operator operates the input unit 41 to input information on the ultraviolet irradiation dose (set time and intensity) to be irradiated to the affected area. Next, the operator holds the gripping unit 23 of the treatment tool and brings the radiation surface 25 into contact or proximity to the affected area.

The operator then presses the button 27a of the indication unit 27 provided on the gripping unit 23. Then, UV LEDs 24a are turned on and ultraviolet irradiation to the affected area is started.

Then, when the ultraviolet irradiation reaches the input irradiation dose (and the input irradiation time), UV LEDs 24a automatically turn off.

As explained above, the present invention makes it possible to provide the ultraviolet therapy apparatus that uses LEDs as the light source that can achieve at least one of the same or better therapeutic effects and the same or worse side effects compared to conventional ultraviolet therapy apparatus that uses excimer lamps as the light source.

The ultraviolet therapy apparatus according to the present invention is not limited to the above embodiments, and various changes can be made on it.

1. An ultraviolet therapy apparatus comprising a light source unit that emits ultraviolet light, the light source unit comprising at least one LED, the LED being configured to:
in a case in which an integral intensity of an emission spectrum in a wavelength range of 250 nm to 400 nm is 1,
have a ratio of an integral intensity in wavelengths of 250 nm to 298 nm to an integral intensity in the wavelength range is less than or equal to 0.088; and
emit an emission spectrum such that a ratio of an integral intensity of wavelengths of 308 nm to 313 nm to an integral intensity of wavelengths of 250 nm to 298 nm is greater than or equal to 5.2.

2. An ultraviolet therapy apparatus comprising a light source unit that emits ultraviolet light, the light source unit comprising at least one LED, the LED being configured to:
in a case in which an integral intensity of an emission spectrum in a wavelength range of 250 nm to 400 nm is 1,
have an intensity at a wavelength of 298 nm is less than or equal to 0.0078;
have an intensity at a wavelength of 295 nm is less than or equal to 0.0055;
have an intensity at a wavelength of 290 nm is less than or equal to 0.0033;
have an intensity at a wavelength of 280 nm is less than or equal to 0.0015; and
emit an emission spectrum such that a ratio of an integral intensity of wavelengths of 308 nm to 313 nm to an integral intensity of wavelengths of 250 nm to 298 nm is greater than or equal to 5.2.

3. An ultraviolet therapy apparatus comprising a light source unit that emits ultraviolet light, the light source unit comprising at least one LED, the LED being configured to:
in an erythema ultraviolet spectrum E_{CIE} in a wavelength range of 250 nm to 400 nm,
emit an emission spectrum such that a ratio of an integral intensity of wavelengths of 250 nm to 298 nm to an integral intensity of the wavelength range is less than or equal to 0.44,
wherein the erythema ultraviolet spectrum E_{CIE} is represented as
$${E}_{CIE} = {B}_{λ} \times {S}_{e r} / ( {\int }_{250}^{400} {E}_{λ} d λ )$$
where E_{λ} is a spectral irradiance of the ultraviolet light emitted from the LED, and Sₑᵣ is an erythema action spectrum and represented as
$${S}_{e r} ( λ ) = \{ \begin{matrix}1 & ( 250 nm < λ < 298 nm ) \\ 1 {0}^{0.094 ( 298 - λ )} & ( 298 nm ≦ λ ≦ 328 nm ) \\ 1 {0}^{0.015 ( 139 - λ )} & ( 328 nm < λ < 400 nm ) .\end{matrix}$$

4. An ultraviolet therapy apparatus comprising a light source unit that emits ultraviolet light, the light source unit comprising at least one LED, the LED being configured to:
in an erythema ultraviolet spectrum E_{CIE} in a wavelength range of 250 nm to 400 nm,
emit an emission spectrum such that a ratio of an integral intensity of wavelengths of 250 nm to 298 nm to an integral intensity of the wavelength range is less than or equal to 0.44,
wherein the erythema ultraviolet spectrum E_{CIE} is represented as
$${E}_{CIE} = P \times {S}_{e r}$$
where P is an emission spectrum of the LED, area normalized in a wavelength range of 250 nm to 400 nm, and Sₑᵣ is an erythema action spectrum and represented as
$${S}_{e r} ( λ ) = \{ \begin{matrix}1 & ( 250 nm < λ < 298 nm ) \\ 1 {0}^{0.094 ( 298 - λ )} & ( 298 nm ≦ λ ≦ 328 nm ) \\ 1 {0}^{0.015 ( 139 - λ )} & ( 328 nm < λ < 400 nm ) .\end{matrix}$$

5. The ultraviolet therapy apparatus according to claim 3, the apparatus is configured to emit an emission spectrum such that a ratio of an integral value of wavelengths of 308 nm to 313 nm to an integral value of wavelengths of 250 nm and 298 nm is greater than or equal 0.47 in the erythema ultraviolet spectrum E_{CIE}.

6. The ultraviolet therapy apparatus according to claim 1, wherein a peak wavelength of an emission spectrum of the LED is between 308 nm and 313 nm.

7. The ultraviolet therapy apparatus according to claim 1, wherein a full width at half maximum of an emission spectrum of the LED is less than or equal to 20 nm.

8. The ultraviolet therapy apparatus according to claim 1, wherein a spectrum on an irradiated surface is the spectrum recited in claim 1.

9. A light source for emitting ultraviolet light, the light source comprising at least one LED, the LED being configured to:
in a case in which an integral intensity of an emission spectrum in a wavelength range of 250 nm to 400 nm is 1,
have a ratio of an integral intensity in wavelengths of 250 nm to 298 nm to an integral intensity in the wavelength range is less than or equal to 0.088; and
emit an emission spectrum such that a ratio of an integral intensity of wavelengths of 308 nm to 313 nm to an integral intensity of wavelengths of 250 nm to 298 nm is greater than or equal to 5.2.

10. A light source for emitting ultraviolet light, the light source comprising at least one LED, the LED being configured to:
in a case in which an integral intensity of an emission spectrum in a wavelength range of 250 nm to 400 nm is 1,
have an intensity at a wavelength of 298 nm is less than or equal to 0.0078;
have an intensity at a wavelength of 295 nm is less than or equal to 0.0055;
have an intensity at a wavelength of 290 nm is less than or equal to 0.0033;
have an intensity at a wavelength of 280 nm is less than or equal to 0.0015; and
emit an emission spectrum such that a ratio of an integral intensity of wavelengths of 308 nm to 313 nm to an integral intensity of wavelengths of 250 nm to 298 nm is greater than or equal to 5.2.

11. A light source for emitting ultraviolet light, the light source comprising at least one LED, the LED being configured to:
in an erythema ultraviolet spectrum E_{CIE} in a wavelength range of 250 nm to 400 nm,
emit an emission spectrum such that a ratio of an integral intensity of wavelengths of 250 nm to 298 nm to an integral intensity of the wavelength range is less than or equal to 0.44,
wherein the erythema ultraviolet spectrum E_{CIE} is represented as
$${E}_{CIE} = {E}_{λ} \times {S}_{e r} / ( {\int }_{250}^{400} {E}_{λ} d λ )$$
where E_{λ} is a spectral irradiance of the ultraviolet light emitted from the LED, and Sₑᵣ is an erythema action spectrum and represented as
$${S}_{e r} ( λ ) = \{ \begin{matrix}1 & ( 250 nm < λ < 298 nm ) \\ 1 {0}^{0.094 ( 298 - λ )} & ( 298 nm ≦ λ ≦ 328 nm ) \\ 1 {0}^{0.015 ( 139 - λ )} & ( 328 nm < λ < 400 nm ) .\end{matrix}$$

12. A light source for emitting ultraviolet light, the light source comprising at least one LED, the LED being configured to:
in an erythema ultraviolet spectrum E_{CIE} in a wavelength range of 250 nm to 400 nm,
emit an emission spectrum such that a ratio of an integral intensity of wavelengths of 250 nm to 298 nm to an integral intensity of the wavelength range is less than or equal to 0.44,
wherein the erythema ultraviolet spectrum E_{CIE} is represented as
$${E}_{CIE} = P \times {S}_{e r}$$
where P is an emission spectrum of the LED, area normalized in a wavelength range of 250 nm to 400 nm, and Sₑᵣ is an erythema action spectrum and represented as
$${S}_{e r} ( λ ) = \{ \begin{matrix}1 & ( 250 nm < λ < 298 nm ) \\ 1 {0}^{0.094 ( 298 - λ )} & ( 298 nm ≦ λ ≦ 328 𝔫m ) \\ 1 {0}^{0.015 ( 139 - λ )} & ( 328 nm < λ < 400 nm ) .\end{matrix}$$

13. The light source according to claim 11, the light source is configured to emit an emission spectrum such that a ratio of an integral value of wavelengths of 308 nm to 313 nm to an integral value of wavelengths of 250 nm and 298 nm is greater than or equal 0.47 in the erythema ultraviolet spectrum E_{CIE}.

14. The light source according to claim 9, wherein a peak wavelength of an emission spectrum of the LED is between 308 nm and 313 nm.

15. The light source according to claim 9, wherein a full width at half maximum of an emission spectrum of the LED is less than or equal to 20 nm.
